(19) United States
(12) Patent Application Publication
(10) Pub. No.: US 2021/0102256 A1
(43) Pub. Date: Apr. 8, 2021

(54) SYSTEMS AND METHODS FOR PREDICTING AND MONITORING CANCER THERAPY

(71) Applicant: PREDICINE, INC., HAYWARD, CA (US)

(72) Inventors: Shidong JIA, Palo Alto, CA (US); Pan DU, Dublin, CA (US); Zhixin ZHAO, Mountain View, CA (US)

(21) Appl. No.: 16/499,449

(22) PCT Filed: Mar. 30, 2018
(86) PCT No.: PCT/US18/25335

§ 371 Date: Sep. 30, 2019

Related U.S. Application Data
(63) Continuation-in-part of application No. PCT/US17/52832, filed on Sep. 21, 2017.
Application No. PCT/US17/52832 is a continuation-in-part of application No. PCT/US17/27830, filed on Apr. 16, 2017.
(60) Provisional application No. 62/480,278, filed on Mar. 31, 2017.
(60) Provisional application No. 62/397,923, filed on Sep. 21, 2016.
(60) Provisional application No. 62/480,288, filed on Mar. 31, 2017.
(60) Provisional application No. 62/323,547, filed on Apr. 15, 2016.

(51) Int. Cl. C12Q 1/6886

(52) U.S. Cl. CPC C12Q 1/6886; G01N 2800/52; C12Q 2600/158; C12Q 2600/154

(57) ABSTRACT

Disclosed are systems and methods for diagnosing a disease status or progression of a disease comprising detection and correlation of methylation and expression level changes of cancer markers including PD-L1 in a biofluid sample from the patient. The systems and methods are similarly applicable to the detection and correlation of methylation and expression level changes in solid tissues. The present method utilized a barcoding method for analysis. The systems and methods can be used for predicting immunotherapy resistant cancer and monitoring therapy response.

### FIELD OF INVENTION

The invention relates generally to the field of precision medicine, specifically cancer prediction, diagnostics or prognostics, and more specifically of methods for predicting cancer development in cancer patients by the detection of methylation and gene expression of essential genes for cancer treatment using human biofluid samples, e.g., plasma, urine, CSF and saliva.

### BACKGROUND

Cancer immunotherapy is a treatment that involves components of the immune system in human bodies. Most immunotherapies use antibodies which bind to the proteins expressed by cancer cells, therefore, inhibiting the protein function. Previously, FDA has approved inhibitors of either the programmed death receptor (PD-1) or its ligand (PD-L1) for treatment of cancer patients with metastatic melanoma, metastatic non-small cell lung cancer, recurrent or metastatic head and neck cancer and urothelial carcinoma. In 2017, FDA approved the pembrolizumab (anti-PD-1 antibody) for any unresectable or metastatic solid tumor with microsatellite instability (MSI) or mismatch repair deficiency (MMR).

PD-1 (CD279) is primarily expressed on the surface of activated T-cells and PD-L1 (CD274) is the main ligand of PD-1. PD-1/PD-L1 binding inhibits the function of activated T-cells. High level of PD-L1 from the cancer cells helps cancer cells evade immune attack by binding PD-1 and inhibiting of T-cell activation. One important clinical question regarding the PD-1/PD-L1 directed immunotherapy is the uncertainty of the clinical outcome. Currently, the most popular biomarker for anti-PD-1/PD-L1 therapy is PD-L1 detection by immunohistochemistry (IHC). Companion diagnostic PD-L1 IHC tests for anti-PD-1 therapy have been approved by FDA (PD-L1 IHC 22C3 pharmDx, DAKO/AGILENT TECHNOLOGIES).

However, one key limitation of using IHC is the amount of tissue obtained from the patient. Meanwhile, tissue heterogeneity also needs to be considered for the accuracy of IHC. A liquid biopsy diagnosis is an elegant and non-invasive way to discover genetic alterations. More recently, next generation sequencing (NGS) is accelerating the discovery of genetic and epigenetic alteration in human diseases. One of the major advantages of next generation sequencing is multiplex sequencing. NGS technology also enables adding a molecular barcode to identify the source of the NGS reads.

### SUMMARY OF THE INVENTION

Disclosed are systems and methods for predicting and monitoring cancer immunotherapy response in a patient. The present invention provides a method for diagnosing a disease status or progression of a disease comprising 1) assaying the methylation and expression status of one or more cancer markers in a biofluid sample from a subject, and 2) detecting the presence of one or more methylation sites of the cancer marker and level of RNA expression of the cancer marker; wherein the correlation of the methylation level and RNA expression changes indicates the disease status. The present invention further discloses a method for diagnosing a disease status or progression of a disease comprises 1) assaying the methylation and expression status of one or more cancer markers in a biofluid sample from a subject, and 2) assaying presence or absence of the correlation of the methylation level of the cancer marker to level of RNA expression of the cancer marker; wherein the presence of one or more methylation sites and the presence of correlation of the methylation level and RNA expression changes indicates the disease status. The cancer markers include but are not limited to PD-L1 or other cancer markers known in the art. The disease status including the presence or absence of the disease, the progress of the disease, the prediction of the disease. The present invention provides an accurate diagnosis than other known tools.

In another aspect, the method employs an approach to isolate RNA from circulating tumor RNA (ctRNA) and RNA-containing extracellular vesicles in a biofluid sample. The biofluid sample can be a sample of blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, or a combination thereof.

In one aspect, the disclosed method comprises assaying the methylation and expression status of PD-L1 gene in a biofluid sample from the patient; wherein the presence of one or more CG methylation sites and low PD-L1 expression indicates the reduced immunotherapy output. In one aspect, the means for assaying the methylation and expression status of PD-L1 gene comprises a) extracting cell-free nucleic acid from a biofluid sample of the patient, b) bisulfite treatment of DNA, c) barcoding the DNA and RNA, and d) bioinformatics assaying of the barcoded DNA and RNA related to PD-L1 gene.

In one aspect, the method is for predicting immunotherapy in cancer patients. In one aspect, the present invention disclosed a method of combined DNA and RNA assays comprising a step of bioinformatics analysis identifying genetic and epigenetic biomarker signatures that enables prospective prediction of therapies in a cancer patient. In some embodiments, the method comprises assaying the presence or absence of CpG methylation of cancer markers in a biofluid sample from the patient; wherein the presence of the one or more CpG methylation site and low PD-L1 RNA expression level indicate the presence of poor clinical outcome of the anti-PD-1/PD-L1 therapy in cancer patients.

Also disclosed is a kit for detecting methylation and expression of cancer markers in a patient, comprising: (a) reagents for circulating nucleic acid extraction; (b) reagents for bisulfite conversion; and (c) reagents for reverse transcription; (d) reagents for library preparation; (e) reagents for MSP and/or ddPCR; (f) and/or oligos targeting cancer marker genes.

In some embodiments, the kit is used for detecting CpG methylation and expression of cancer marker genes by NGS.

In some embodiments, the kit is used for detecting CpG methylation and expression of PD-L1 by NGS.

In other embodiments, the kit is used for detecting specific CpG methylation site and gene expression of cancer markers by ddPCR.

Disclosed are systems and methods for detecting genetic and epigenetic alterations in lung, breast, ovarian, prostate and other cancer patients. In one aspect, the disclosed method comprises assaying the presence or absence of one or more epigenetic alteration such as DNA methylation and presence or absence of one or more genetic alterations such as gene splice variants, mutation, indels, long deletions, copy number variation, gene fusions etc. in a biofluid sample from the said patient.

The present invention further disclosed the system for detecting a genetic and epigenetic alteration from a biofluid sample comprises: a) obtaining cell-free nucleic acids from the biofluid and preparing two portions of sample wherein one portion comprises single strand RNA (ssRNA) and the other portion comprises a double strand DNA(dsDNA), b) barcoding ssRNA and converting ssRNA to ds-cDNA wherein the ds-cDNA is a barcoded ds-cDNA, and c) treating the dsDNA with bisulfite and converting the dsDNA to two ssDNA with mutations of Cytosine to Uracil. d) converting the mutated ssDNA to mutated dsDNA wherein the mutated dsDNA are barcoded dsDNA, e) loading the barcoded ds-cDNA and the barcoded dsDNA portions to NGS sequencer for further genetic alteration analysis.

The present invention further discloses Gene RADAR (RNA and DNA single molecular digital Reading) assay and bioinformatics analysis tool for combined genetics and epigenetics alteration analysis. The bioinformatics analysis enables 1) differentiation of the RNA derived reads from DNA derived reads by checking the RNA specific tags in the sequence reads; 2) the suppression of the sequencing and background noise by creating consensus of Next Generation Sequence (NGS) reads from the same original molecules, which is defined based on molecular barcodes and the mapping location of the reads; and 3) accurate quantification of RNA by combining two types of barcodes (RNA specific molecular barcodes+DNA molecular barcodes), and the quantification of DNA (only using DNA molecular barcodes) at the same time; and 4) detection of epigenetic alteration by quantification of DNA methylation.

### BRIEF DESCRIPTION OF THE DRAWINGS

FIG. 1 shows an assay flowchart depicting the steps from plasma nucleic acid extraction to NGS and ddPCR assay of methylation and gene expression.

FIG. 2A-2D show the negative correlation between PD-L1 methylation and gene expression. FIG. 2A shows PD-L1 DNA methylation and mRNA expression level are negatively correlated based on TCGA data. Using sample 1 and sample 2 as materials, we performed ddPCR assay for PD-L1 gene expression (FIG. 2B) and methylation NGS assay for methylation status of PD-L1 (FIG. 2C) and the results showed the methylation and expression of PD-L1 in these two samples are negatively correlated (FIG. 2D).

### DETAILED DESCRIPTION OF THE INVENTION

The present invention provides a method for diagnosing a disease status or progression of a disease comprising 1) assaying the methylation and expression status of one or more cancer markers in a biofluid sample from a subject, and 2) detecting the presence of one or more methylation sites of the cancer marker gene and level of RNA expression of the cancer marker; wherein the correlation of the methylation level and RNA expression changes indicates the disease status. The cancer marker genes include but are not limited to DNA, variations, genetic alterations, modifications and any changes made but remain the main characteristics of the gene itself.

The present invention disclosed a method for diagnosing a disease status or progression of a disease comprises 1) assaying the methylation and expression status of one or more cancer markers in a biofluid sample from a subject, and 2) assaying presence or absence of the correlation of the methylation level of the cancer marker to level of RNA expression of the cancer marker; wherein the presence of one or more methylation sites and the presence of correlation of the methylation level and RNA expression changes indicates the disease status. The cancer markers include but are not limited to PD-L1 or other cancer markers known in the art. The disease status including the presence or absence of the disease, the progress of the disease, the prediction of the disease. The present invention can also be combined with other known tools to aid in the determination of the disease status.

In one aspect, the disease includes but not limiting to tumor, cancer or other diseases that related to the cancer makers.

In another aspect, the disease status includes but not limiting to the stage of the disease, the progress of the disease.

In yet another aspect, the diagnosing a disease includes but not limiting to the detecting a disease, detecting a disease status by using companion diagnostics method, detecting the disease status by using a condition including but not limiting to drug treatment or using other means.

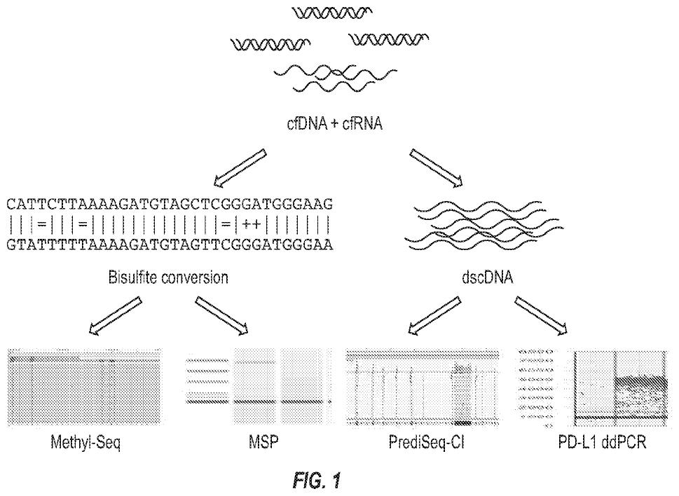

The present invention discloses a novel method for identifying genetic and epigenetic biomarker signatures that enables prospective prediction of resistance to anti-PD-1/PD-L1 therapies in a cancer patient. The present invention also disclosed a gene panel of multiple mixed genes for predicting and monitoring cancer immunotherapy. FIG. 1 shows the flowchart including the steps from plasma nucleic acid extraction to NGS and/or ddPCR assay.

In one aspect, the present disclosure allows the detection of methylation and expression of interesting genes from biofluids in specimens of various sources. There are abundant ctRNA and extracellular vesicles in cancer patients and the abundance of ctRNA and extracellular vesicles are independent of the number of CTCs. Hence, the present method enables the detection of genetic alterations in more patients than CTC-based methods. In some embodiments, the disclosed method streamlines the extraction of circulating DNA and RNA from biofluids, bisulfite conversion of the DNA, reverse transcription of the RNA and NGS library preparation and sequencing. In contrast to existing methods known in the art, this streamlined process enables the non-invasive detection of methylation and gene expression of interest by using biofluid samples in a quick, simplified, and consistent manner.

In some embodiments, the present disclosure provides a method for detecting various solid cancers in a patient comprising assaying the presence or absence of CpG methylation site of PD-L1 and expression of interesting immunotherapy genes in a biofluid sample from the patient.

In some embodiments, the step of assaying comprises extracting RNA from the biofluid sample and subsequently reverse transcribing the extracted RNA into a complementary DNA (cDNA).

In other embodiments, the step of assaying comprises extracting both DNA and RNA from the biofluid sample simultaneously and then bisulfate convert the extracted DNA into single strand DNA (ssDNA).

In some embodiments, the resultant ssDNA is subsequently used to detect the PD-L1 methylation by Methylation-Specific Polymerase Chain Reaction (MSP).

In some embodiments, the resultant ssDNA is subsequently measured by Next Generation Sequencing and/or Polymerase Chain Reaction.

In some embodiments, the resultant cDNA is subsequently used to detect the PD-L1 expression by digital droplet Polymerase Chain Reaction (ddPCR).

In some embodiments, the resultant cDNA is subsequently measured by Next Generation Sequencing. In some embodiments, the resultant cDNA is subsequently measured by Polymerase Chain Reaction.

The system can be a closed system and an automated system.

### Definitions

The term “about,” particularly in reference to a given quantity, is meant to encompass deviations of plus or minus five percent.

As used in this application, including the appended claims, the singular forms “a,” “an,” and “the” include plural references, unless the content clearly dictates otherwise, and are used interchangeably with “at least one” and “one or more.”

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

The term “patient,” as used herein preferably refers to a human, but also encompasses other mammals. It is noted that, as used herein, the terms “organism,” “individual,” “subject,” or “patient” are used as synonyms and interchangeably.

The term “genetic alteration” comprise gene splice variants, SNV, Indel, CNV, fusion and combination thereof.

The term “epigenetic alteration” comprising DNA and RNA methylation.

The term “gene expression” means transcription and/or translation of genes. Specifically herein, gene expression is the transcription of DNA to RNA.

The term circulating tumor DNA (ctDNA) or circulating tumor RNA (ctRNA) is tumor-derived fragmented DNA or RNA in the bloodstream that is not associated with cells. ctDNA or ctRNA should not be confused with cell-free DNA (cfDNA) or cell-free RNA (cfRNA), a broader term which describes DNA or RNA that is freely circulating in the bloodstream, but is not necessarily of tumor origin.

The term cell-free DNA (cfDNA) or cell-free RNA (cfRNA) is DNA or RNA freely circulating in the bloodstream and not associated with cells.

The term “barcoding” or “barcode” means using one or more oligonucleotides as tags/markers to incorporate into a dsDNA. The barcodes will be sequenced together with the unknown sample DNA. After sequencing the reads are sorted by barcode and grouped together (de-multiplexing). Barcode includes molecular barcode and sample barcode.

A “molecular barcode” is a unique multiple-base pair sequence used to identify unique fragments and “de-duplicate” the sequencing reads from a sample. This, along with the random start sites, helps identify and remove PCR duplicates. Molecular barcodes can be used to suppress sequencing and PCR errors, and reduce false positives subsequently. Whereas sample barcodes, also called indexed adaptors, are customarily used in most current NGS workflows and allow the mixing of samples prior to sequencing.

The term “RNA molecular barcoding” means incorporating barcodes during the process of reverse transcription of RNA and ds-cDNA library preparation. RNA molecular barcoding can incorporate a molecular barcode or multiple molecular barcodes. An RNA specific barcode can be an RNA specific tag, a molecular barcode, a sample barcode or a combination.

The term “DNA barcoding” means barcoding at dsDNA level with a multiple-base pair sequence that is part of the adapter for multiplex sequencing. In some embodiment, the adapter is designed in-house. The incorporated DNA barcodes can be molecular barcodes alone or both molecular barcodes and sample barcodes.

The term “positive” strand also known as the “sense” strand or coding strand, is the segment within double-stranded DNA that runs from 5′ to 3′. The term “negative” strand also known as the “anti-sense” strand of DNA is the segment within double-stranded DNA that runs from 3′ to 5′.

The term “bioinformatics” means a sequencing analysis tool/software including but are not limited to Gene RADAR software or any software that can analyze DNA/RNA sequencing results.

“Cancer markers” include but are not limited to PD1, PD-L1 and PD-L2, human stratum corneum chymotryptic enzyme (HSCCE), kallikrein 4, kallikrein 5, kallikrein 6 (protease M), kallikrein 8, kallikrein 9, kallikrein 10, CA125, CA15-3, CA19-9, OVX1, lysophosphatidic acid (LPA), carcinoebryonic antigen (CEA), macrophage colonystimulating factor (M-CSF), prostasin, CA54-61, CA72, HMFG2, IL-6, IL-10, LSA, M-CSF, NB70K, PLAP, TAG72, TNF, TPA, UGTF, WAP four-disulfide core domain 2 (HE4), matrix metalloprotease 2, tetranectin, inhibin, mesothelyn, MUC1, VEGF, CLDN3, NOTCH3, E2F transcription factor 3 (E2F3), GTPase activating protein (RAC GAP1), hematological and neurological expressed 1 (HN1), apolipoprotein A1, laminin, claudin 3, claudin 4, tumorassociated calcium signal transducer 1 (TROP-1/Ep-CAM), tumor-associated calcium signal transducer 2 (TROP-2), ladinin 1, S100A2, SERPIN2 (PAI-2), CD24, lipocalin 2, matriptase (TADG-15), stratifin, transforming growth factor-beta receptor III, platelet-derived growth factor receptor alpha, SEMACAP3, ras homology gene family member I(ARHI), thrombospondin 2, disabled-2/differentially expressed in ovarian carcinoma 2 (Dab2/DOC2), and haptoglobin-alpha subunit, MCR-A61, MCR-6A3, MCR-573, MCR-A42, MCR-425, and MCR-CBE, KLK3-SERPINA3, EGFR, BMPER, FGA/FGB/FGG, C9, STX1A, AKR7A2, CKB/CKM, DDC, CA6, IGFBP2, IGFBP4, FN1, BMP1, CRP, KIT, CNTN1, SERPINA1, BDNF, GHR, ITIH4, NME2, AHSG, 6Ckine, ACE, BDNF, E-Selectin, EGF, Eot2, ErbBl, follistatin, HCC4, HVEM, IGF-11, IGFBP-1, IL-17, IL-lsrll, IL-2sRa, leptin, M-CSFR, MIF, MIP-la, MIP3b, MMP-8, MMPI, MPIF-1, OPN, PARC, PDGF Rb, prolactin, ProteinC, TGF-b RIii, TNF-R1, TNFa, VAP-1, VEGF R2 and VEGF R3.

The present invention provides a method for barcoding an oligonucleotide tag on the RNA sample during reserve transcribing it to cDNA and ds-cDNA, wherein the reverse transcription step of the ssRNA includes 1) reverse transcribing ssRNA to cDNA using a gene-specific or random primer annealed to an oligonucleotide comprising a RNA specific tag and random molecular barcodes; and/or 2) converting the cDNA to a ds-cDNA. In some embodiments, converting the cDNA to a ds-cDNA is conducted by annealing a non-coded primer, wherein such barcoding step is named single-sided RNA barcoding. In one embodiment, the RNA specific tag comprising an oligonucleotide. The random molecular barcodes comprise another oligonucleotide. The oligonucleotide consists of 5, 8, 10, 12, 14, 15, 20 nucleic acid bases. In another embodiment, the oligonucleotide can be designed for fitting the identification in further analysis.

In some other embodiments, the converting step of the cDNA to a ds-cDNA is conducted by annealing an oligonucleotide comprising a second RNA specific molecular barcode, wherein such barcoding step is named double-sided RNA barcoding. In some embodiment, the first and second RNA specific molecular barcode is the same; in some other embodiment, the first RNA and second RNA specific molecular barcode is not the same.

In preferred embodiments, the genetic alterations include gene splice variants, mutations, indels, long deletions, copy number changes, fusions and combination thereof. The method of detecting the alterations is used to detect the changes of above.

In one embodiment, the barcoded signals from RNA and DNA are read by next generation sequencing. Separation of RNA and DNA derived reads is conducted with the Gene RADAR bioinformatics analysis tool.

In another embodiment, after the next generation sequencing simultaneous reads signals from RNA and DNA, a database file of the RNA molecular barcodes and DNA molecular barcodes will be utilized to recognize the reads from RNA or DNA with barcode matches. Then the recognized DNA reads are mapped to the genome, while the recognized RNA reads are mapped to transcriptome and genome. Barcode consensus is created by merging NGS reads originally from the same molecule (identified based on molecular barcodes and genome mapping location of the reads). The sequencing and PCR errors can be corrected or marked when there are inconsistent variants originally from the same molecule. In some embodiments, the genetic alteration of DNA includes SNV, Indel, long deletion, CNV and DNA fusion. In some embodiments, the genetic alteration of RNA includes splicing, fusion, SNV, Indel analysis. In some embodiments, the epigenetic alteration of DNA includes methylation of one or more CpG sites. Then the DNA and RNA analysis results are integrated to achieve comprehensive reporting of genetic and epigenetic alterations.

In one embodiment, barcode or barcoding with d oligonucleotide sequences such as 5, 8, 10 12, 14, 15 nucleotides to uniquely tag individual target DNA molecules can be used. In another embodiment, the oligonucleotide can be designed for fitting the identification in further analysis. Such application increases the sensitivity and reduces false positives. For example, it can be used for PCR or NGS analysis to identify individual molecules (DNA or RNA fragments) in samples.

In some embodiments, the Gene RADAR detects DNA methylation at CpG sites and DNA level variants while measuring other RNA expression and RNA level variants including splicing, fusion, SNV, Indel at the same time from the patient biofluid sample.

The present invention further provides a platform for detecting multiple gene variants in a patient, including: (a) a kit of reagents for circulating nucleic acid extraction; (b) barcoding sequences for two-layer RNA+DNA molecular barcoding; and (c) bioinformatics tool to analyzing DNA and RNA-derived information.

The system can be an opened or closed system. And both systems can be an automated system. The system can be in a device setting.

In preferred embodiments, the detection of presence or absence of a genetic alteration and epigenetic alteration is indicative of the immunotherapy resistance to a disease and the disease is one or more cancers. In some other embodiments, presence or absence of multiple genetic alteration and presence/absence of one or more epigenetic alteration is indicative of a disease and the disease is one or more cancers. In some other embodiments, presence/absence of multiple epigenetic alteration and presence/absence of one or more genetic alteration are indicative of a disease and the disease is one or more cancers.

In one embodiment, the samples include but are not limited to blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, and a combination thereof.

In some embodiments, the detected genetic alteration and epigenetic alteration information can be used to detect immunotherapy-resistant cancer in a patient comprising assaying the presence or absence of one or more types of genetic alterations and epigenetic alterations at both RNA and DNA levels, such as PD-L1 gene DNA methylation and RNA/DNA-based mutation detection in a biofluid sample from the patient; wherein the presence of such genetic and epigenetic alterations indicates the presence of immunotherapy-resistant cancer in the patient.

In some embodiments, RNA is extracted together with DNAs from circulating nucleic acid and nucleic acid-containing extracellular vesicles in a biofluid sample.

In some embodiments, the sources of nucleic acids are extracellular vesicles (EVs), including exosomes and microvesicles, which have been shown to carry a variety of biomacromolecules including mRNA, microRNA and other non-coding RNAs and considered to be a minimally invasive novel source of materials for molecular diagnostics. See Jia et al., “Emerging technologies in extracellular vesicle-based molecular diagnostics”, Expert Rev. Mol. Diagn. 1-15 (2014). EVs are membranous, cell-derived, mixed populations of vesicles, ranging from approximately 40-5000 nm in diameter, which are released by a variety of cells into the intercellular microenvironment and various extracellular biofluids. Methods for procuring a microvesicle fraction from a biofluid sample are described in scientific publications and patent applications (Chen et al., 2010; Miranda et al., 2010; Skog et al., 2008). See also WO 2009/100029, WO 2011009104, WO 2011031892, and WO 2011031877. For example, methods of microvesicle procurement by differential centrifugation are described in a paper by Raposo et al. (Raposo et al., 1996), a paper by Skog et al. (Skog et al., 2008) and a paper by Nilsson et. al. (Nilsson et al., 2009). Methods of anion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS) is described in a paper by Taylor and Gercel-Taylor (Taylor and Gercel-Taylor, 2008). A method of nanomembrane ultrafiltration concentration is described in a paper by Cheruvanky et al. (Cheruvanky et al., 2007). Further, microvesicles can be identified and isolated from a subject's bodily fluid by a microchip technology that uses a microfluidic platform to separate tumor-derived microvesicles (Chen et al., 2010).

Methods for nucleic acid extraction are generally based on procedures well-known in the art plus proprietary procedures developed in-house. Persons of skill will select a particular extraction procedure as appropriate for the particular biological sample. Examples of extraction procedures are provided in patent publications WO/2009/100029, US 20100196426, US 20110003704, US 20110053157, WO 2011009104, WO 2011031892, US20130131194 and US20110151460. Each of the foregoing references is incorporated by reference herein for its teaching of these methods.

Many biofluids contain circulating nucleic acids and/or nucleic acid-containing EVs. Examples of these biofluids include blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, or a combination thereof.

In some embodiments, the biofluid sample is obtained from a subject who has been diagnosed with cancer according to tissue or liquid biopsy and/or surgery or clinical grounds.

It must be noted that, as used in this specification and the appended claims, the singular forms “a”, “an” and “the” include plural referents unless the content clearly dictates otherwise. Thus, for example, a reference to “a biomarker” includes a mixture of two or more biomarkers, and the like.

### EXAMPLES

### Example 1

Extracting cell-free Nucleic Acid (cfNA) from human bio-fluid samples, e.g., plasma, urine and saliva and using Gene RADAR technology to split the extracted the cfNA as cfDNA and cfRNA.

About 5-100 ng cfDNA was used to obtain single-strand DNA(ssDNA) by DNA bisulfite treatment. Subsequently the ssDNA can be used as a template for methylation-specific PCR (MSP) of PD-L1; the ssDNA can also be used for an ssDNA library preparation and a small portion of the ssDNA library can be used for PD-L1 MSP. The ssDNA library can be enriched using Methylation panel, which contains the CpG methylation sites of PD-L1 gene.

Separately cfRNA was reverse transcribed into single-strand cDNA. So the cDNA can be used as a template for PD-L1 ddPCR; the cDNA can also be used for cDNA library preparation and a small portion of the cDNA library can be used for PD-L1 ddPCR. The cDNA library can be enriched using Cancer immunotherapy panel of mixed genes.

Enriched ssDNA and cDNA libraries are barcoded with different barcodes and loaded on sequencer for NGS.

DNA and RNA data are analyzed for DNA methylation and RNA expression respectively.

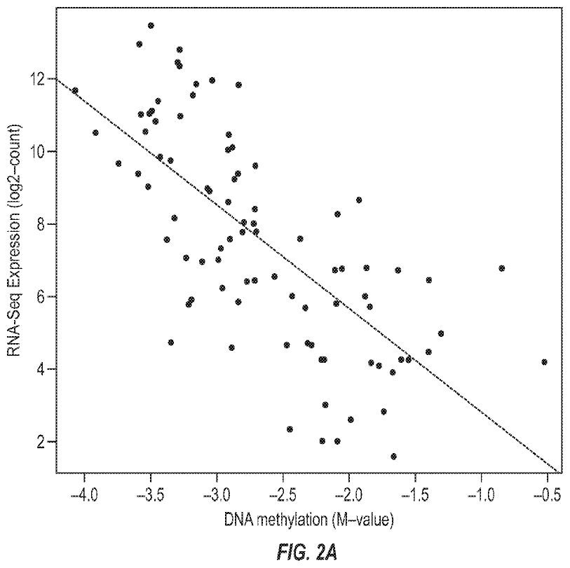

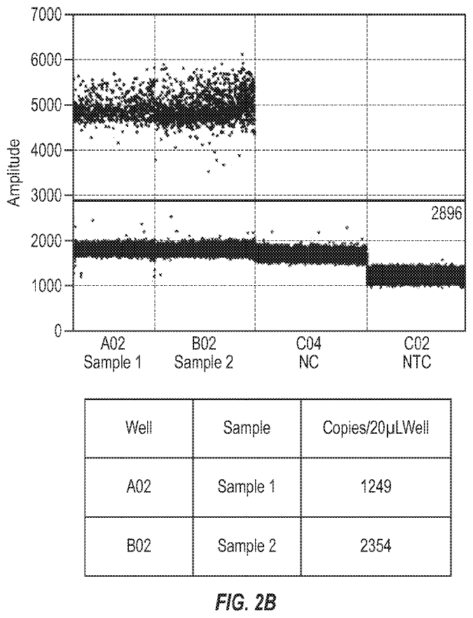

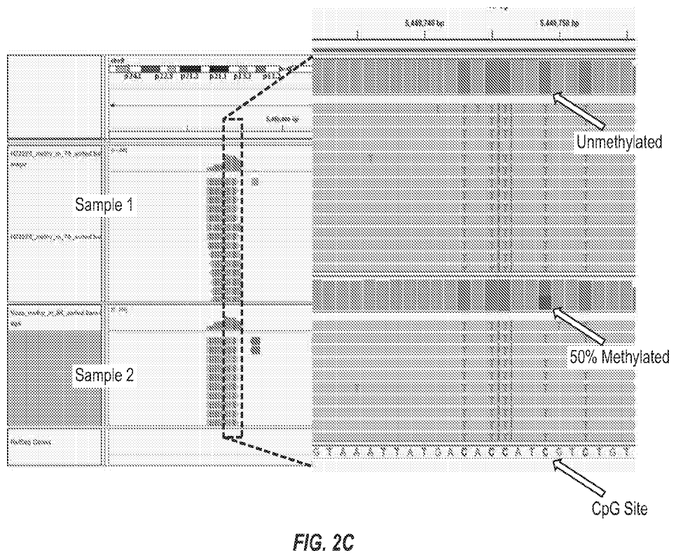

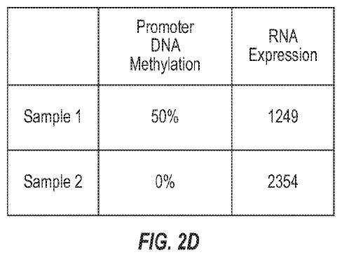

Using sample 1 and sample 2 from patient's bio-fluid as materials, we performed ddPCR test for both PD-L1 RNA expression (FIG. 2B) and methylation NGS test for methylation status of PD-L1 (FIG. 2C). The results showed that the methylation and expression of PD-L1 in these two samples are negatively correlated (FIG. 2D). These negative correlations indicate that combined assaying of DNA methylation and RNA expression in bio-fluid samples is a reliable way to predict the treatment response of patients.

The disclosures of all publications, patents, patent applications and published patent applications referred to herein by an identifying citation are hereby incorporated herein by reference in their entirety. Websites references using “World-Wide-Web” at the beginning of the Uniform Resource Locator (URL) can be accessed by replacing “World-Wide-Web” with “www.”

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

We claim:

1. A method for diagnosing a disease status or progression of a disease comprising 1) assaying the methylation and expression status of one or more cancer markers in a biofluid sample from a subject, and 2) detecting the presence of one or more methylation sites of the cancer marker and level of RNA expression of the cancer marker; wherein the correlation of the methylation level and RNA expression changes indicates the disease status.

2. The method of claim 1, wherein the biofluid sample is a sample of blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, or a combination thereof.

3. The method of claim 1, wherein the assaying the methylation and expression status of one or more cancer marker gene comprises a step of extracting RNA from the biofluid sample and subsequently reverse transcribing the extracted RNA into a complementary DNA.

4. The method of claim 1, wherein the assaying comprised extracting both DNA and RNA from the same biofluid sample and then bisulfate convert the extracted DNA into single strand DNA (ssDNA).

5. The method of claim 4, wherein the resultant ssDNA is subsequently used to detect the cancer marker methylation by Methylation-Specific Polymerase Chain Reaction (MSP).

6. The method of claim 4, wherein the resultant ssDNA is subsequently measured by Next Generation Sequencing and/or Polymerase Chain Reaction.

7. The method of claim 3, wherein the cDNA is subsequently measured by Next Generation Sequencing, Polymerase Chain Reaction, and/or array-based technologies.

8. A method for predicting immunotherapy in cancer patients comprising assaying the presence or absence of CpG methylation of PD-L1 and assaying PD-L1 expression level in a biofluid sample from a subject; wherein the presence of the one or more CpG methylation sites and low PD-L1 RNA expression level indicate the presence of poor clinical outcome of the anti-PD-1/PD-L1 therapy in cancer patients.

9. A system for detecting a genetic and epigenetic alteration of a cancer marker from a biofluid comprises:
a) obtaining nucleic acids from the biofluid and preparing two portions of sample wherein one portion comprises a single strand RNA (ssRNA) and the other portion comprises a double strand DNA(dsDNA),
b) barcoding the ssRNA and converting the ssRNA to a ds-cDNA wherein the ds-cDNA is a barcoded ds-cDNA, and
c) treating the dsDNA with bisulfite and converting the dsDNA to two ssDNA with mutations of Cytosine to Uracil,
d) converting the mutated ssDNA to mutated dsDNA wherein the mutated dsDNA are barcoded dsDNA,
e) loading the barcoded ds-cDNA and the barcoded dsDNA portion for further genetic alteration analysis.

10. The system of claim 9, wherein the system is a closed system.

11. The system of claim 9, wherein the system is an automated system.

12. A kit for detecting methylation and expression of a cancer marker, comprising: (a) reagents for circulating nucleic acid extraction; (b) reagents for bisulfite conversion; (c) reagents for reverse transcription; (d) reagents for library preparation; (e) reagents for MSP and/or ddPCR; and/or (f) oligos targeting cancer marker genes.

13. The kit of claim 12, wherein the kit is used for detecting CpG methylation and expression of PD-L1 genes by NGS.

14. The kit of claim 12, wherein the kit is used for detecting CpG methylation and expression of cancer marker by NGS.

15. The kit of claim 12, wherein the kit is used for detecting specific CpG methylation site and gene expression of cancer marker by ddPCR.

16. A method for simultaneous detection of a genetic and epigenetic alteration from a biofluid comprises:
a) obtaining cell free nucleic acids (cfNA) from the biofluid and preparing two portions of sample wherein one portion comprises a single-strand RNA (ssRNA) and the other portion comprises a double strand DNA(dsDNA),
b) converting unmethylated cytosine in dsDNA to uracil and denaturing dsDNA to single-strand DNA (ssDNA) by DNA bisulfite treatment,
c) enriching ssDNA derived from methylation panel gene,
d) barcoding ssRNA with a first barcode during reverse transcription step and converting ssRNA to ds-cDNA wherein the ds-cDNA is a barcoded ds-cDNA,
e) enriching single-strand cDNA derived from immunotherapy panel gene,
f) barcoding ssDNA with a second barcode, converting ssDNA to dsDNA wherein the dsDNA is a barcoded dsDNA,
g) loading the barcoded dsDNA portion and the barcoded ds-cDNA portion to sequencer of NGS for further genetic alteration analysis.

17. The method of claim 16, wherein the reverse transcription step of the barcoded ssRNA comprises 1) reverse transcribing ssRNA to cDNA after ssRNA is annealed to an oligonucleotide comprising an RNA specific tag, and random molecular barcodes; and 2) converting the cDNA to a ds-cDNA.

18. The method of claim 16, wherein the genetic alteration comprising one or more gene splice variants, mutations, indels, copy number changes, fusions and combination thereof.

19. The method of claim 16, wherein the biofluid sample is selected from a group consisting of blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, and a combination thereof.

20. The method of claim 16, wherein the barcoded DNA mixture is subsequently analyzed by Next Generation Sequencing, Polymerase Chain Reaction, and/or array-based technologies.

21. The method of claim 16, wherein the detection of presence and absence of a genetic alteration is indicative of a disease.

22. The method of claim 16, wherein the disease is cancer.

23. The method of claim 16, wherein the methylation panel comprising the CpG methylation sites of cancer marker gene.

24. The method of claim 16, wherein the immunotherapy panel comprising one or more cancer marker gene.

25. A method for diagnosing a disease status or progression of a disease comprising 1) assaying the methylation and expression status of one or more cancer markers in a biofluid sample from a subject, and 2) assaying presence or absence of the correlation of the methylation level of the cancer marker to level of RNA expression of the cancer marker; wherein the presence of one or more methylation sites and the presence of correlation of the methylation level and RNA expression changes indicates the disease status.
